(19) United States
(12) Patent Application Publication
(10) Pub. No.: US 2007/0148197 A1
(43) Pub. Date: Jun. 28, 2007

(54) Treating inflammation with ondansetron and pharmaceutical compositions comprised thereof

(76) Inventors: Fabrizio Dolfi, Valbonne (FR); Nadege Tremel, Villeneuve Loubet (FR)

Correspondence Address: BUCHANAN, INGERSOLL & ROONEY PC, POST OFFICE BOX 1404, ALEXANDRIA, VA, 22313-1404, US

(21) Appl. No.: 11/450,390

(22) Filed: Jun. 12, 2006

Related U.S. Application Data
(63) Continuation of application No. PCT/EP04/13841, filed on Dec. 6, 2004.

(30) Foreign Application Priority Data
May 26, 2004 (EP) 04291328.5

(51) Int. Cl. A61K 31/4178

(52) U.S. Cl. 424/401; 514/397

(57) ABSTRACT

Inflammatory skin disorders such as rosacea, including a variety of skin diseases, whether or not accompanied by dermatosis, are treated by topically applying onto the affected skin area, a thus effective amount of Ondansetron, or salt or base thereof.

### CROSS-REFERENCE TO PRIORITY/PCT/PROVISIONAL APPLICATIONS

This application claims priority under 35 U.S.C. § 119 of Provisional Application No. 60/528,587, filed Dec. 10, 2003, EP 04291328.5, filed May 26, 2004, and is a continuation of PCT/EP 2004/013841 filed Dec. 6, 2004 and designating the United States, published in the English language as WO 2005/058312 A1 on Jun. 30, 2005, each hereby expressly incorporated by reference and each assigned to the assignee hereof.

### BACKGROUND OF THE INVENTION

1. Technical Field of the Invention

The present invention generally relates to the administration of ondansetron as an active principle formulated into pharmaceutical compositions, more particularly dermatological compositions, suited for a curative and/or prophylactic anti-inflammatory treatment, via the topical route. More specifically, the present invention relates to the administration of Ondansetron to topically treat rosacea, an inflammatory skin disorder. By “Ondansetron” is intended ondansetron, its ionic salts, for example hydrochloride salt, or its non-ionic base forms.

2. Description of Background and/or Related and/or Prior Art

Ondansetron, or (i) 1,2,3,9-tetrahydro-9-methyl-3-[(2-methyl-1H-imidazol-1-yl)methyl]-4H-carbazol4-one, is a compound which is already known per se, and is disclosed and claimed in U.S. Pat. No. 4,695,578. See also The Merck Index, 13th Edition, page 6919. Ondansetron acts as a potent and selective antagonist of the neuronal 5HT receptor type 3, located on primary afferent nerves. Specifically, Ondansetron has been approved in the past in palliative therapy to prevent or treat chemotherapy induced nausea and vomiting. In addition, this compound is useful as an analgesic, for example, in the alleviation of pain associated with migraine, headache, and many other forms of pain for which 5HT is the endogenous mediator.

No published data exist regarding the use of ondansetron, by the topical route, to treat the inflammatory skin condition, rosacea.

### SUMMARY OF THE INVENTION

Thus, the present invention features the administration of an anti-inflammatory effective amount of ondansetron formulated into a topical pharmaceutical composition for the treatment of inflammation. Additionally, the present invention features a regime or regimen of treating inflammation by topically applying a pharmaceutical composition comprising ondansetron. More specifically, the present invention features the administration of ondansetron to topically treat rosacea, an inflammatory skin disorder.

### DETAILED DESCRIPTION OF BEST MODE AND SPECIFIC/PREFERRED EMBODIMENTS OF THE INVENTION

Rosacea is a common, but often overlooked, skin condition of uncertain etiology that can lead to significant facial disfigurement, ocular complications, and severe emotional distress. The progression of rosacea is variable; however, typical stages include: (1) facial flushing, in which mostly the cheeks appear to have a flush or sunburn, the redness being caused by dilation of the blood vessels in the skin, which allows more blood to flow and pool under the surface of the skin, (2) erythema and/or edema, telangiectasia, (3) pimples appearing as small red and solid (papules) or pus-filled (pustules), and (4) rhinophyma.

Central facial erythema and telangiectasis are predominant in the early stages of rosacea. This progresses to a chronic inflammatory infiltrate with central facial papules and, less commonly, pustules. Intermittent or chronic facial edema may also occur. Some patients develop rhinophyma, a coarse hypertrophy of the connective tissue and sebaceous glands of the nose evident as small knobby bumps on the nose.

The predominant, presenting complaints, conditions or afflictions of rosacea are intermittent, central facial flushing and erythema. Itching is typically absent; however, many patients complain of a stinging pain (which can be severe) associated with flushing episodes. These flushing episodes are often socially embarrassing and can occur unpredictably or can be linked to environmental, chemical, food, or emotional triggers. Common triggers include exposure to the sun, cold weather, sudden emotion (laughter or embarrassment), hot beverages, and alcohol consumption.

According to the present invention, topical ondansetron (in cream, gel, or lotion form, for example) administered once or twice daily is effective for facial flushing. The present invention also indicates that topical ondansetron can reduce pustular and papular rosacea, and erythema and telangiectasis in some patients.

As a consequence, the present invention features the topical application of an effective amount of ondansetron formulated into a topical pharmaceutical composition for treating rosacea. This invention also features the administration of ondansetron for the treatment of rosacea where the rosacea is treated at a stage in which facial flushing is evident, and preferably, where the rosacea is treated at a stage in which a symptom selected from the group consisting of erythema, edema, telangiectasia and ocular is evident.

Rosacea is a chronic, relapsing disorder, and long-term treatment is generally required. Control of symptoms can be successfully maintained by long-term topical use of ondansetron.

Other characteristics, aspects, objectives and advantages of the invention will become still more clearly apparent from the description which follows and various specific, but in no way limiting, examples intended to illustrate same.

Preferably, the pharmaceutical compositions of the invention are intended for topical use. More particularly, the pharmaceutical composition is a dermatological composition.

Such a composition according to the present invention is a composition for the topical treatment of inflammation comprising an anti-inflammatory effective amount of ondansetron and a topical pharmaceutically acceptable carrier therefor.

The dermatological composition is more generally suited for the treatment, by the topical route, of skin diseases or complaints, conditions or afflictions having at least a vascular lesion, one inflammatory component or, at the same time, one inflammatory and one infectious component.

More particularly, the skin diseases or complaints, conditions or afflictions correspond to skin inflammations accompanying any type of dermatosis such as eczema, psoriasis, rosacea, acne vulgaris, ulcers, seborrhoeic dermatitis and irritations induced by chemical, physical or mechanical agents or others. The present invention is especially useful in the topical treatment of rosacea by administering ondansetron.

In what follows, “topical route” means any technique for administration of a product by direct application to a surface (or external) part of the body, such as the skin or eye.

By the topical route, the pharmaceutical compositions based on ondansetron, which are therefore more particularly suited for the treatment of the skin or mucous membranes, can be provided in the form of ointments, creams, milks, salves, powders, impregnated pads, solutions, gels, sprays, lotions or suspensions. They can also be provided in the form of lipid or polymer vesicles or nanospheres or microspheres or in the form of polymer patches or hydrogels making possible a controlled release formulation. These compositions by the topical route can moreover be provided either in anhydrous form or in an aqueous form, according to the clinical indication. Formulations for topical application which are particularly highly suitable in the context of the implementation of the present invention are given in particular in the examples of the present specification.

In a preferred embodiment of the invention, the topical composition is in the form of a gel, a cream or a lotion.

By the ocular route, compositions of the present invention are mainly eyewashes.

The compositions, preferably for topical use, according to the invention contain ondansetron at a concentration preferably of from 0.01% to 5% by weight, more preferably 0.5, and most preferably 0.75% with respect to the total weight of the composition.

According to a specific and preferred embodiment of the present invention, the overall content of the ondansetron does not exceed 5 to 10% of the total weight of the composition. Preferably, the medicinal compositions are topically applied twice a day, for a period of 3 months.

The medicinal compositions according to the invention can, of course, additionally contain inert or even pharmacodynamically or cosmetically active additives or combinations of these additives and in particular: wetting agents; depigmenting agents such as hydroquinone, azelaic acid, caffeic acid or kojic acid; emollients; hydrating agents such as glycerol, PEG 400, thiamorpholinone and its derivatives or, alternatively, urea; anti-seborrhoeic or anti-acne agents, such as S-carboxymethylcysteine, S-benzylcysteamine, their salts or their derivatives, or benzoyl peroxide; fungicides such as ketoconazole or 4,5-polymethylene-3-isothiazolidones; carotenoids and in particular beta-carotene; anti-psoriatic agents such as anthralin and its derivatives; and finally 5,8,11,14-eicosatetraynoic acid and 5,8,11-eicosatriynoic acid and their esters and amides; metronidazole, ivermectine, and other agents used for treating rosacea; anti-inflammatory agents such as NSAIDS.

The compositions according to the invention can also contain flavor-improving agents, preservatives, such as the esters of para-hydroxybenzoic acid, stabilizing agents, moisture-regulating agents, pH-regulating agents, agents for modifying osmotic pressure, emulsifying agents, UV-A and UV-B screening agents, and antioxidizing agents, such as alpha-tocopherol, butylated hydroxyanisole or butylated hydroxytoluene.

Of course, one skilled in this art will take care to choose the optional compound(s) to be added to the pharmaceutical composition so that the advantageous properties intrinsically associated with the composition are not, or not substantially, detrimentally affected by the envisaged addition.

A number of examples intended to illustrate various specific formulations in accordance with the invention will now be given, it being understood that same are intended only as illustrative and in nowise limitative. In said examples to follow, all parts and percentages are given by weight, unless otherwise indicated.

### EXAMPLE 1

This example is of a specific formulation in accordance with the invention which is provided in the form of a gel for topical use.

| | | |
|---|---|---|
| Ondansetron | 0.75 | g |
| Carbopol 980 (Goodrich) | 0.6 | g |
| Polyethylene glycol 400 | 3 | g |

| | |
|---|---|
| Sodium hydroxide | q.s. pH 5 |
| Preservatives | q.s. |
| Demineralized water | q.s. for 100 g |

### EXAMPLE 2

This example is of a specific formulation in accordance with the invention which is provided in the form of a cream for topical use.

| | | |
|---|---|---|
| Ondansetron | 0.75 | g |
| Methyl glucose sesquistearate | 1 | g |
| Stearyl alcohol | 0.5 | g |
| Liquid paraffin oil | 6 | g |
| Polyethylene glycol 400 | 2 | g |
| Methyl glucose sesquistearate | 5 | g |
| polyoxyethylenated with 20 mol of EO | | |
| Carbopol 981 (Goodrich) | 0.4 | g |
| Glycerol | 7 | g |
| Cyclomethicone | 4 | g |

| | |
|---|---|
| Sodium hydroxide | q.s. pH 5 |
| Preservatives | q.s. |
| Demineralized water | q.s. for 100 g |

### EXAMPLE 3

This example is of a specific formulation in accordance with the invention which is provided in the form of a lotion for topical use.

| | |
|---|---|
| Ondansetron | 0.75 g |
| Benzyl alcohol | 1.30 |
| Glycerol | 7.00 |
| Stearyl alcohol | 2.00 |
| Light mineral oil | 6.00 |
| Carbomer 941 | 0.15 |
| Glyceryl Stearate | 3.00 |
| Potassium Sorbate | 0.20 |
| Cyclomethicone | 4.00 |
| PEG-8 | 2.00 |
| Steareth-21 | 3.00 |
| Lactic acid | qs pH adjustment |
| Sodium Hydroxyde | qs pH adjustment |
| Purified Water | qs 100.00 |

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for the treatment of an inflammatory skin disorder, comprising topically applying onto the affected skin area of an individual in need of such treatment, for such period of time as required to elicit the desired effect, a thus effective amount of Ondansetron, or salt or base thereof.

2. The regime or regimen as defined by claim 1, for the treatment of a skin disease.

3. The regime or regimen as defined by claim 1, for the treatment of a skin disease accompanied by dermatosis.

4. The regime or regimen as defined by claim 3, for the treatment of eczema, psoriasis, acne rosacea, acne vulgaris, ulcers, seborrhoeic dermatitides or irritations induced by chemical, physical or mechanical agents.

5. The regime or regimen as defined by claim 4, for the treatment of rosacea.

6. The regime or regimen as defined by claim 5, for the treatment of rosacea at a stage in which facial flushing is evident.

7. The regime or regimen as defined by claim 5, for the treatment of rosacea at a stage in which erythema, edema, telangiectasia or an ocular symptom is evident.

8. A topically applicable pharmaceutical/dermatological composition useful for the treatment of inflammatory skin disorders, comprising a thus effective amount of Ondansetron, or salt or base thereof, formulated into a topically applicable, pharmaceutically acceptable carrier therefor.

9. The topically applicable pharmaceutical/dermatological composition as defined by claim 8, comprising from 0.01% to 5% by weight of Ondansetron, or salt or base thereof.

10. The topically applicable pharmaceutical/dermatological composition as defined by claim 8, formulated as a gel.

11. The topically applicable pharmaceutical/dermatological composition as defined by claim 8, formulated as a cream.

12. The topically applicable pharmaceutical/dermatological composition as defined by claim 8, formulated as a lotion.

13. The topically applicable pharmaceutical/dermatological composition as defined by claim 9, comprising from 0.5% to 0.75% by weight of Ondansetron, or salt or base thereof.
